(19) United States
(12) Patent Application Publication
(10) Pub. No.: US 2007/0196841 A1
(43) Pub. Date: Aug. 23, 2007

(54) PHYSIOGENOMIC METHOD FOR PREDICTING RESPONSE TO DIET

(76) Inventors: Gualberto Ruano, Milford, CT (US); Andreas Windemuth, Woodbridge, CT (US); Jeff Scott Volek, Bolton, CT (US)

Correspondence Address: MORGAN & FINNEGAN, L.L.P., 3 WORLD FINANCIAL CENTER, NEW YORK, NY, 10281-2101, US

(21) Appl. No.: 11/625,107

(22) Filed: Jan. 19, 2007

Related U.S. Application Data
(60) Provisional application No. 60/760,652, filed on Jan. 20, 2006.

(51) Int. Cl. C12Q 1/68; C07H 21/04

(52) U.S. Cl. 435/6; 536/23.1

(57) ABSTRACT

The present invention relates to the use of genetic variants of associated marker genes to predict an individual's response to diet. The present invention further relates to analytical assays and computational methods using the novel marker gene set. The present invention has utility for developing personalized diet regimens to optimize physiological response, including changes in body mass index (BMI) and blood lipid and triglyceride levels.

### FIELD OF THE INVENTION

The present invention is in the field of physiological genomics, hereafter referred to as “physiogenomics”. More specifically, the invention relates to the use of genetic variants of marker genes to predict an individual's responsiveness to diet. The invention also relates to methods for generating patient-specific physiotype models for expressing predicted effects of diet on Body Mass Index (BMI) and HDL cholesterol, LDL cholesterol, and triglyceride levels.

### BACKGROUND OF THE INVENTION

It has recently been estimated that the obesity rate among the adult population of the United States has doubled in the past two decades to a staggering 31% [1]. During the same time, the percentage of adults that are either overweight or obese rose to 65% [1]. This dramatic rise in obesity has led to a public health crisis owing to the increased prevalence of disease with increased body weight. For example, overweight and obesity increases the risk of developing cardiovascular disease, cancer, diabetes, high blood pressure, elevated cholesterol, and stroke, among other serious conditions.

Despite the public health imperative of obesity, the morbidity and mortality associated with obesity are largely preventable with lifestyle and dietary changes. For example, a recent report by the American Institute for Cancer Research and the world Cancer Research Fund estimated that 30-40% of cancer cases worldwide are preventable by diet [2]. Other studies report that progression to diabetes in pre-diabetics may be reduced by 40-58% through lifestyle intervention, including dietary modification [3].

Many well known diets exist which emphasize restriction of various dietary components. For example, the latest recommendations call for a diet that is high in carbohydrates and low in total fat, saturated fat and cholesterol [4]. However, it is an alarming reality that low-fat/high-carbohydrate diets actually exacerbate the co-morbidity of obesity, diabetes, and cardiovascular disease, a condition now recognized as Metabolic Syndrome (MetSyn) which is characterized as having 3 or more of the following abnormalities: (1) large waist circumference (>102 cm in men, 88 cm in women), (2) elevated serum triglycerides (>150 mg/dL), (3) depressed high density lipoprotein (HDL, <40 mg/dL in men, 50 mg/dL in women), (4) elevated blood pressure (systolic >130 mm Hg or diastolic ≧80 mmHg), and (5) elevated serum glucose (>110 mg/dL) [4]. A primary problem with low-fat/high-carbohydrate diets is that they contribute to carbohydrate-induced hypertriglyceridemia [5], a major problem underlying the metabolic disorders of MetSyn. Well-controlled feeding studies indicate that low-fat/high-carbohydrate diets exacerbate the dyslipidemia of MetSyn when not associated with significant weight loss or increased physical activity [6,7]. Low-fat/high-carbohydrate diets have unfavorable effects on fasting triglycerides [8], HDL-C [9], and size and composition of LDL-C [10,11]. Clearly, the standard low-fat recommendations are not suited for all individuals and may in fact be counterproductive to dieting goals.

Recently, carbohydrate-restricted diets have gained increased popularity. In this regard, very-low-carbohydrate “ketogenic” diets (VLCKDs) have proven effective in combating obesity for many individuals. VLCKDs differ dramatically from the standard recommendations by emphasizing a reduction in carbohydrates and thus are inherently high in total fat, saturated fat, and cholesterol. For this reason, VLCKDs have been criticized as having potential adverse effects on blood lipoproteins and other risk factors for cardiovascular disease and diabetes [12]. However, these criticisms are largely unsupported and based on a misunderstanding of the physiological adaptations to carbohydrate restriction. Recent studies have demonstrated that short-term VLCKDs consistently result in improvements in fat-loss and a number of cardiovascular disease factors as compared to low-fat diets [12]. Nonetheless, substantial variability in cholesterol and blood lipid responses to VLCKDs has been reported [13].

It may therefore be said that it is impossible to create a singular optimal diet for everyone. This is because of the complex interactions among nutrition, environment, and, importantly, genetics. A genetic explanation for variability in response to diet has been shown in studies that measured polymorphisms of selected candidate genes, usually apolipoproteins. APOE and APOA1 predict cholesterol responses to diet [14]. An increase in polyunsaturated fat increases HDL-C in individuals carrying the A allele at the −75 G/A genetic polymorphism of the APOA1 gene, whereas those with the more common G allele decrease HDL-C [15]. The response of HDL-C to increases in total fat, particularly animal fat, is explained in part by a polymorphism in the hepatic lipase gene [16]. While intriguing, genetic variations in single genes are not precise in their predictive power. More robust approaches that consider many genes and the multidimensional interactions with various phenotypes are needed. Thus, despite the recognition of the importance of genomics in personalized nutrition [17,18,19,20], there are currently no viable methods to guide personalized diet prescription.

The emerging field of physiogenomics offers an important approach for integrating genotype, phenotype, and population analysis of functional variability among individuals. In physiogenomics, genetic markers (e.g. single nucleotide polymorphisms or “SNPs”) are analyzed to discover statistical associations to physiological characteristics or outcomes in populations of individuals either at baseline or after they have been exposed to an environmental trigger such as dietary intervention.

It is therefore an object of the present invention to provide physiogenomic methods and tools for predicting the response of individuals to diet based on genetic factors, alone or in combination with demographic factors.

It is another object of the invention to provide genetic markers and arrays of genetic markers which are predictive of BMI, triglyceride, and blood lipid response to diet.

It is a further object of the invention to provide combinations of genetic markers which are more predictive of BMI, triglyceride, and blood lipid response to diet than the individual markers.

### SUMMARY OF THE INVENTION

In accordance with the foregoing objectives and others, the present invention provides physiogenomic methods and tools for a priori determination of an individual's likely response to diet. The method utilizes physiogenomics to identify gene variants, in particular single nucleotide polymorphisms (SNPs), which correlate with changes in BMI, LDL, HDL, and triglyceride levels in response to diet.

In one aspect, the present invention provides marker gene sets comprising a plurality of single nucleotide polymorphic gene variants, wherein the presence of any one of said single nucleotide polymorphic gene variants in a human is predictive of physiological response to diet. The physiological response may be, for example, change in blood triglyceride level, change in blood LDL level, change in blood HDL level, change in body mass index, or any combination of these responses.

In one interesting implementation according to this aspect of the invention, the plurality of single nucleotide polymorphic gene variants will comprise at least one single nucleotide polymorphic gene variant of a gene selected from the groups consisting of ABCB1, ACACB, ACAT1, ACHE, ADRB1, ADRB2, AKT1, AKT2, ANGPT1, APOB, APOH, APOL3, APOL4, AVEN, BDNF, CETP, CHAT, CHKB, CPT1A, CRHR2, CYP1A2, CYP2C19, CYP7A1, DBH, DRD3, DRD4, DRD5, DTNBP1, FLJ32252, FLT1, GABRA2, GAD1, GAD2, GAL, GNAO1, GYS2, HIF1A, HTR3A, ICAM1, IL10, IL1R1, INSR, IRS1, KDR, LDLR, LIPE, LIPF, LOC391530, OLR1, OXT, PIK3C2G, PIK3C3, PIK3R1, PPARG, PRKAA1, PRKAB1, RARB, RARG, RXRA, SCARB2, SELE, SSTR3, VEGF, and combinations thereof.

Particularly interesting single nucleotide polymorphisms of the foregoing genes are selected from the group consisting of rs10082776, rs1018381, rs1040410, rs10422283, rs1042713, rs1042718, rs1045642, rs10460960, rs1062688, rs1064344, rs107540, rs10841044, rs10890819, rs11212515, rs1128503, rs1150226, rs1190762, rs1290443, rs132642, rs132661, rs1478290, rs167771, rs1801278, rs1801701, rs1951795, rs2005590, rs2033447, rs2049045, rs2071710, rs2125489, rs2l92752, rs2240403, rs2241220, rs2301108, rs2306179, rs2429511, rs2470890, rs2494746, rs2514869, rs2702285, rs2742115, rs2743867, rs2867383, rs3024492, rs322695, rs3750546, rs3756007, rs3757868, rs3791850, rs3791981, rs3792822, rs3808607, rs3813065, rs3853188, rs4135268, rs4244285, rs4531, rs461404, rs4802071, rs4804103, rs4986894, rs4987059, rs5030390, rs5361, rs563895, rs5880, rs5883, rs597316, rs619698, rs6265, rs694066, rs706713, rs748253, rs8110695, rs814628, rs8178847, rs8190586, rs833060, rs877172, and rs885834.

In another implementation of the invention, the marker gene set is predictive of change in blood LDL level and the single nucleotide polymorphic gene variants comprise one or more single nucleotide polymorphic gene variants selected from the group consisting of rs1018381, rs4804103, or both.

In another implementation of the invention, the marker gene set is predictive of change in blood HDL level and the single nucleotide polymorphic gene variants comprise one or more single nucleotide polymorphic gene variants selected from the group consisting of rs1064344, rs3756007, rs8110695, rs4244285, rs3024492, rs2192752, rs2514869, rs1190762, and combinations thereof.

In yet another implementation of the invention, the marker gene set is predictive of change in blood triglyceride level and the single nucleotide polymorphic gene variants comprise one or more single nucleotide polymorphic gene variants selected from the group consisting of rs132642, rs3757868, rs1951795, rs3791981, rs10460960, and combinations thereof.

In a further implementation of the invention, the marker gene set is predictive of change in body mass index and the single nucleotide polymorphic gene variants comprise one or more single nucleotide polymorphic gene variants selected from the group consisting of rs814628, rs4531, rs2306179, rs4987059, rs5883, rs5361, rs877172, and combinations thereof.

Another specific aspect of the method involves obtaining genetic material, e.g. DNA or RNA, from a subject, and assaying the genetic material to determine if any of the single nucleotide polymorphic gene variants belonging to the marker gene set are present, wherein the presence of the one or more single nucleotide polymorphic gene variants is predictive of physiological response to diet. Micro- and nano-array analysis of the subject's genetic material is preferred in this specific aspect of the invention.

In another aspect, the present invention further provides a method for the development of novel diagnostic systems, termed “physiotypes”, which are developed from combinations of gene polymorphisms and baseline characteristics, to provide physicians with individualized patient response profiles for physiological response to diet.

Yet another aspect of the present invention provides a system containing a support or support material, e.g. a micro- or nano-array, comprising a novel set of marker genes and/or gene variants associated with physiological response to diet in a form suitable for the practitioner to employ in a screening assay for determining an individual's genotype. In addition to the marker genes and gene variants, the system comprises an algorithm for predicting the physiological response to diet based on a predetermined set of mathematical equations providing specific coefficients to each of the components of the array.

In another aspect, the present invention provides methods for the identification of a population of individuals that will respond favorably to diet, including but not limited to carbohydrate-restricted diet, based on the physiological responses of change in blood triglyceride level, change in blood LDL level, change in blood HDL level, change in body mass index, or any combination of these responses. These individuals, who are identified through screening using the methods of the present invention, are especially amenable to carbohydrate-restricted diet to reduce weight and reduce the occurrence of obesity related morbidity and mortality.

These and other aspects of the present invention will be better understood upon a reading of the following detailed description when considered in connection with the accompanying figures.

### BRIEF DESCRIPTION OF THE DRAWINGS

FIG. 1. Distribution of HDL change, with the reference range indicated.

FIG. 2. Individual genotypes (circles) of SNPs overlaid on the distribution of HDL change (thin line). Each circle represents a subject, with the horizontal axis specifying the HDL change, and the vertical axis the genotype: bottom—homozygous for major allele, middle—heterozygous, top—homozygous for minor allele. A LOESS fit of the allele frequency as a function of HDL change (thick line) is shown.

FIG. 3. Individual genotypes (circles) of SNPs overlaid on the distribution of body mass change (BMS) (thin line). Each circle represents a subject, with the horizontal axis specifying the BMS change, and the vertical axis the genotype: bottom—homozygous for major allele, middle—heterozygous, top—homozygous for minor allele. A LOESS fit of the allele frequency as a function of BMS change (thick line) is shown.

FIG. 4. A representational display of an individual patient's predicted response (LDL, HDL, triglycerides, and BMI) to carbohydrate-restricted dieting.

### DETAILED DESCRIPTION OF THE INVENTION

Very low-carbohydrate (VLC) diets have been reported to outperform low-fat diets on a number of metrics, including weight/fat loss and metabolic biomarkers of CVD and diabetes [21-29]. Table 1 summarizes published studies comparing low-fat (LF) and very low-carbohydrate (VLC) diets on percent changes in fasting blood lipids and postprandial lipemia.

TABLE 1

| Reference | Subjects | Wk | Diet | TC | LDL | HDL | TAG | PP TAG |
|---|---|---|---|---|---|---|---|---|
| Volek et al., 2000 [21] | NW Men | 8 | VLC | 1.8 | 10.3 | 10.0 | −54.9 | −48% |
| Sharman et al., 2002 [28] | NW Men | 6 | VLC | 4.7 | 4.2 | 11.5 | −33.0 | −29% |
| Volek et al., 2003 [24] | NW Women | 4 | VLC | 15.8* | 14.6* | 32.0* | −30.2* | −16.0* |
| | | | LF | −5.2 | −4.8 | −7.7 | 3.8 | 9.6% |
| Sharman et al., 2004 [29] | OW Men | 6 | VLC | −10.9 | −6.2* | −3.3 | −44.1* | −37.6* |
| | | | LF | −14.8 | −17.4 | −6.6 | −15.0 | −19.5 |
| Volek et al., 2004b [26] | OW Women | 4 | VLC | 1.1* | 4.6* | 1.3* | −23.0 | −28.9 |
| | | | LF | −7.1 | −5.7 | −8.6 | −11.2 | −24.5 |

*P ≦ 0.05 from corresponding change on LF diet. TC = total cholesterol; TAG = triglyceride; PP = postprandial; NW = normal-weight; OW = overweight

Despite the reported efficacy of VLC diet intervention, the variability in response highlights the desirability of providing individualized dietary regimens to optimize response. This approach necessary requires the consideration of patient genotype. However, the specific contribution of genetic influences to diet response is not well understood. Therefore, it has not previously been possible to provide accurate patient-specific dietary recommendations. It has surprisingly been found that physiogenomic methods can be employed to identify genetic markers associated with response to interventional VLC dieting. A patient can then be assayed for the presence of one or more of the genetic markers and a personalized predicted response profile developed based on the presence or absence of the marker, the specific allele (i.e., heterozygous or homozygous), and the predictive ability of the marker.

The physiogenomics methods employed in the present invention are described generally in U.S. patent application Ser. No. 11/010,716, the contents of which are hereby incorporated by reference. Briefly, the physiogenomics method for predicting whether a particular treatment regimen will produce a beneficial effect on a patient typically comprises (a) selecting a plurality of genetic markers based on an analysis of the entire human genome or a fraction thereof; (b) identifying significant covariates among demographic data and the other phenotypes preferably by linear regression methods (e.g., R² analysis followed by principal component analysis); (c) performing for each selected genetic marker an unadjusted association test using genetic data; (d) using permutation testing to obtain a non-parametric and marker complexity probability (“p”) value for identifying significant markers, wherein the significance is shown by p<0.10, more preferably p<0.05, and even more preferably p<0.01; (e) constructing a physiogenomic model by linear regression analyses and model parameterization for the dependence of said patient's response to treatment with respect to said markers, wherein said physiogenomic model has p<0.10, more preferably p<0.05, and even more preferably p<0.01; and (f) identifying one or more genes not associated with a particular outcome in said patient to serve as a physiogenomic control.

One embodiment of the present invention involves obtaining nucleic acid, e.g. DNA, from a blood sample of a subject, and assaying the DNA to determine the individuals' genotype of one or a combination of the marker genes associated with interventional VLC diet response. Other sampling procedures include but are not limited to buccal swabs, saliva, or hair root. In a preferred embodiment, genotyping is performed using a gene array methodology, which can be readily and reliably employed in the screening and evaluation methods according to this invention. A number of gene arrays are commercially available for use by the practitioner, including, but not limited to, static (e.g. photolithographically set), suspended (e.g. soluble arrays), and self assembling (e.g. matrix ordered and deconvoluted). More specifically, the nucleic acid array analysis allows the establishment of a pattern of gene expression variability from multiple genes and facilitates an understanding of the complex interactions that are elicited in an individual in response to diet.

In a specific embodiment, the array consists of several hundred genes and is capable of genotyping hundreds of DNA polymorphisms simultaneously. Candidate genes for use in the arrays of the present invention are identified by various means including, but not limited to, pre-existing clinical databases and DNA repositories, review of the literature, and consultation with clinicians, differential gene expression models, physiological pathways in metabolism, cholesterol and lipid homeostasis, and from previously discovered genetic associations. In a preferred embodiment, the candidate genes are selected from those shown in Table 2.

TABLE 2

| Gene | Description |
|---|---|
| ABCB1 | ATP-binding cassette, sub-family B (MDR/TAP), member |
| | 1 |
| ACACB | acetyl-Coenzyme A carboxylase beta |
| ACAT1 | acetyl-Coenzyme A acetyltransferase 1 (acetoacetyl |
| | Coenzyme A thiolase) |
| ACHE | acetylcholinesterase (YT blood group) |
| ADRB1 | adrenergic, beta-1-, receptor |
| ADRB2 | adrenergic, beta-2-, receptor, surface |
| AKT1 | v-akt murine thymoma viral oncogene homolog 1 |
| AKT2 | v-akt murine thymoma viral oncogene homolog 2 |
| ANGPT1 | angiopoietin 1 |
| APOB | apolipoprotein B (including Ag(x) antigen) |
| APOH | apolipoprotein H (beta-2-glycoprotein I) |
| APOL3 | apolipoprotein L, 3 |
| APOL4 | apolipoprotein L, 4 |
| AVEN | apoptosis, caspase activation inhibitor |
| BDNF | brain-derived neurotrophic factor |
| CETP | cholesteryl ester transfer protein, plasma |
| CHAT | choline acetyltransferase |
| CHKB | Choline Kinase Beta |
| CPT1A | carnitine palmitoyltransferase 1A |
| CRHR2 | Corticotropin-releasing hormone receptor 2 |
| CRHR2 | corticotropin releasing hormone receptor 2 |
| CYP1A2 | cytochrome P450, family 1, subfamily A, polypeptide 2 |
| CYP2C19 | cytochrome P450, family 2, subfamily C, polypeptide 19 |
| CYP7A1 | cytochrome P450, family 7, subfamily A, polypeptide 1 |
| DBH | dopamine beta-hydroxylase (dopamine |
| | beta-monooxygenase) |
| DRD3 | dopamine receptor D3 |
| DRD4 | dopamine receptor D4 |
| DRD5 | dopamine receptor D5 |
| DTNBP1 | dystrobrevin binding protein 1 |
| FLJ32252 | hypothetical protein FLJ32252 |
| FLT1 | fms-related tyrosine kinase 1 (vascular endothelial growth |
| | factor/vascular permeability factor receptor) |
| GABRA2 | gamma-aminobutyric acid (GABA) A receptor, alpha 2 |
| GAD1 | glutamate decarboxylase 1 (brain, 67 kDa) |
| GAD2 | glutamate decarboxylase 2 (pancreatic islets and brain, |
| | 65 kDa) |
| GAL | galanin |
| GNAO1 | guanine nucleotide binding protein (G protein), alpha |
| | activating activity polypeptide O |
| GYS2 | glycogen synthase 2 (liver) |
| HIF1A | hypoxia-inducible factor 1, alpha subunit (basic |
| | helix-loop-helix transcription factor) |
| HIF1A | Fms-related tyrosine kinase 1 (vascular endothelial growth |
| | factor/vascular permeability factor receptor) |
| HTR3A | 5-hydroxytryptamine (serotonin) receptor 3A |
| ICAM1 | intercellular adhesion molecule 1 (CD54), human rhinovirus |
| | receptor |
| IL10 | interleukin 10 |
| IL1R1 | interleukin 1 receptor, type I |
| INSR | insulin receptor |
| IRS1 | insulin receptor substrate 1 |
| KDR | kinase insert domain receptor (a type III receptor |
| | tyrosine kinase) |
| LDLR | low density lipoprotein receptor (familial |
| | hypercholesterolemia) |
| LIPE | lipase, hormone-sensitive |
| LIPF | lipase, gastric |
| LOC391530 | similar to SALL4B |
| OLR1 | oxidised low density lipoprotein (lectin-like) receptor 1 |
| OXT | Oxytocin (Neurophysin 1) |
| PIK3C2G | phosphoinositide-3-kinase, class 2, gamma polypeptide |
| PIK3C3 | phosphoinositide-3-kinase, class 3 |
| PIK3R1 | phosphoinositide-3-kinase, regulatory subunit 1 (p85 alpha) |
| PPARG | peroxisome proliferative activated receptor, gamma |
| PRKAA1 | protein kinase, AMP-activated, alpha 1 catalytic subunit |
| PRKAB1 | protein kinase, AMP-activated, beta 1 non-catalytic subunit |
| RARB | retinoic acid receptor, beta |
| RARG | retinoic acid receptor, gamma |
| RXRA | retinoid X receptor, alpha |
| SCARB2 | scavenger receptor class B, member 2 |
| SELE | selectin E (endothelial adhesion molecule 1) |
| SSTR3 | somatostatin receptor 3 |
| VEGF | vascular endothelial growth factor |

Each of the foregoing genes, and combinations thereof, are expected to provide useful markers in the practice of the invention. The gene array includes all of the novel marker genes, or a subset of the genes, or unique nucleic acid portions of these genes. The gene array of the invention is useful in discovering new genetic markers of dietary response.

The specific marker will be selected from variants of these genes, or other genes determined to be associated with dietary response. As used herein, the term “variant” refers to mutations, polymorphisms, and insertions and deletions in the nucleic acid sequence of the “wild type” or “normal” gene. Preferred variants in accordance with the invention are single nucleotide polymorphisms (SNPs) which refers to a gene variant differing in the identity of one nucleotide pair from the normal gene. The following table identifies the most promising SNPS, ranked based on the selection criteria of p ≧0.05, for the physiological responses of total cholesterol change (TC), LDL change, HDL change, triglyceride change (TG), log(TG), change in the ratio TC/HDL, change in body mass (BM), change in fat mass (FMS), change in lean body mass (LBM), change in percent fat (PCT), and change in body mass index (BMI).

TABLE 3

| | | | |
|---|---|---|---|
| SNP | p | coeff | Gene |

Total Cholesterol (TC) Change

| | | | |
|---|---|---|---|
| rs3756007 | 0.001303 | 26.03 | GABRA2 |
| rs1018381 | 0.018739 | 16.54 | DTNBP1 |
| rs107540 | 0.021509 | −8.56 | CRHR2 |
| rs461404 | 0.022689 | 10.67 | PRKAA1 |
| rs4804103 | 0.033573 | 29.66 | INSR |
| rs11212515 | 0.037534 | 9.97 | ACAT1 |
| rs5030390 | 0.041028 | 15.55 | ICAM1 |

LDL Change

| | | | |
|---|---|---|---|
| rs3756007 | 0.003566 | 23.47 | GABRA2 |
| rs1018381 | 0.004895 | 19.07 | DTNBP1 |
| rs2743867 | 0.013707 | 18.54 | DTNBP1 |
| rs4804103 | 0.027246 | 30.23 | INSR |
| rs1042718 | 0.039962 | −11.74 | ADRB2 |
| rs1801278 | 0.040607 | 26.72 | IRS1 |
| rs597316 | 0.043344 | 9.26 | CPT1A |

HDL Change

| | | | |
|---|---|---|---|
| rs1064344 | 0.000129 | 6.39 | CHKB |
| rs3756007 | 0.000516 | 6.31 | GABRA2 |
| rs8110695 | 0.013528 | −3.10 | LDLR |
| rs4244285 | 0.015646 | −3.74 | CYP2C19 |
| rs3024492 | 0.017252 | 2.44 | IL10 |
| rs2192752 | 0.018491 | 3.35 | L1R1 |
| rs2514869 | 0.018623 | 4.13 | ANGPT1 |
| rs1190762 | 0.029439 | −5.93 | GNAO1 |
| rs322695 | 0.034483 | −2.64 | RARB |
| rs1150226 | 0.035787 | 7.34 | HTR3A |
| rs2867383 | 0.037263 | −2.26 | DRD5 |
| rs132661 | 0.045862 | −1.93 | APOL3 |
| rs10082776 | 0.046028 | 3.76 | RARG |

Triglycerides (TG) Change

| | | | |
|---|---|---|---|
| rs132642 | 0.001263 | −10.57 | APOL3 |
| rs1951795 | 0.014717 | −16.48 | HIF1A |
| rs3757868 | 0.016743 | 14.76 | ACHE |
| rs8178847 | 0.01754 | 15.94 | APOH |
| rs10841044 | 0.018771 | −15.84 | PIK3C2G |
| rs10460960 | 0.019431 | −19.29 | LOC391530 |
| rs10082776 | 0.032436 | −17.22 | RARG |
| rs706713 | 0.032575 | −10.55 | PIK3R1 |
| rs8190586 | 0.038622 | 17.82 | GAD2 |
| rs833060 | 0.041472 | 9.91 | VEGF |
| rs3791981 | 0.042015 | −16.95 | APOB |
| rs2240403 | 0.045312 | 12.18 | CRHR2 |

Log (TG) Change

| | | | |
|---|---|---|---|
| rs132642 | 0.013774 | −0.11 | APOL3 |
| rs3757868 | 0.014538 | 0.19 | ACHE |
| rs1951795 | 0.020659 | −0.19 | HIF1A |
| rs3791981 | 0.024963 | −0.24 | APOB |
| rs10460960 | 0.026216 | −0.22 | LOC391530 |
| rs2125489 | 0.032805 | −0.36 | KDR |
| rs10841044 | 0.03417 | −0.19 | PIK3C2G |
| rs10082776 | 0.034887 | −0.20 | RARG |
| rs3813065 | 0.043924 | −0.30 | PIK3C3 |

Change in TC/HDL Ratio

| | | | |
|---|---|---|---|
| rs597316 | 0.000234 | 0.56 | CPT1A |
| rs1801701 | 0.001346 | 0.98 | APOB |
| rs2494746 | 0.003759 | 1.06 | AKTI |
| rs2743867 | 0.018089 | 0.60 | DTNBP1 |
| rs1040410 | 0.021294 | 0.65 | DTNBP1 |
| rs5030390 | 0.026579 | 0.60 | CAM1 |
| rs1018381 | 0.027468 | 0.53 | DTNBP1 |
| rs1190762 | 0.033446 | 0.86 | GNAO1 |
| rs11212515 | 0.034032 | 0.35 | ACAT1 |
| rs1062688 | 0.038375 | −0.50 | PRKAB1 |
| rs2514869 | 0.041354 | −0.55 | ANGPT1 |
| rs748253 | 0.048545 | 0.35 | FLT1 |

Body Mass (BMS) Change

| | | | |
|---|---|---|---|
| rs814628 | 0.00022 | −3.66 | LIPF |
| rs5883 | 0.001754 | −2.85 | CETP |
| rs2306179 | 0.006778 | −1.72 | GYS2 |
| rs694066 | 0.023063 | 2.10 | GAL |
| rs2742115 | 0.023702 | 2.17 | OLR1 |
| rs5361 | 0.029515 | −1.52 | SELE |
| rs4531 | 0.031672 | −2.11 | DBH |
| rs3750546 | 0.032014 | 1.38 | RXRA |
| rs2033447 | 0.03817 | 1.11 | RARB |
| rs877172 | 0.042288 | 1.24 | OXT |
| rs3791850 | 0.048218 | −1.77 | GAD1 |

Fat Mass (FMS) Change

| | | | |
|---|---|---|---|
| rs2033447 | 0.003146 | 1.04 | RARB |
| rs5880 | 0.005571 | 2.86 | CETP |
| rs2049045 | 0.00582 | −1.01 | BDNF |
| rs11212515 | 0.007895 | 0.97 | ACAT1 |
| rs6265 | 0.009402 | −1.11 | BDNF |
| rs814628 | 0.010584 | −1.85 | LIPF |
| rs2429511 | 0.010886 | 0.86 | ADRB1 |
| rs2702285 | 0.020131 | 0.97 | AVEN |
| rs563895 | 0.021992 | 1.43 | AVEN |
| rs322695 | 0.027547 | 0.99 | RARB |
| rs2005590 | 0.029808 | −0.71 | APOL4 |
| rs619698 | 0.033252 | −0.98 | FLJ32252 |
| rs3808607 | 0.041825 | 0.87 | CYP7A1 |
| rs885834 | 0.043757 | 0.68 | CHAT |
| rs167771 | 0.044078 | 1.06 | DRD3 |
| rs1042718 | 0.045943 | −0.87 | ADRB2 |

Lean Body Mass (LBM) Change

| | | | |
|---|---|---|---|
| rs3853188 | 0.003288 | 2.82 | SCARB2 |
| rs3750546 | 0.004919 | 1.06 | RXRA |
| rs4804103 | 0.007679 | −2.63 | INSR |
| rs2306179 | 0.007961 | −0.99 | GYS2 |
| rs8178847 | 0.011639 | 1.34 | APOH |
| rs1290443 | 0.011783 | 0.93 | RARB |
| rs2867383 | 0.018063 | −0.63 | DRD5 |
| rs4802071 | 0.019489 | 0.68 | AKT2 |
| rs2742115 | 0.030163 | 1.14 | OLR1 |
| rs1042713 | 0.034098 | −0.96 | ADRB2 |
| rs4135268 | 0.037902 | −1.83 | PPARG |
| rs3756007 | 0.039662 | 1.16 | GABRA2 |
| rs3791850 | 0.042773 | −1.04 | GAD1 |
| rs3792822 | 0.043066 | 0.85 | PRKAA1 |

Change in Percentage Fat

| | | | |
|---|---|---|---|
| rs2049045 | 0.001253 | −1.01 | BDNF |
| rs322695 | 0.002333 | 1.14 | RARB |
| rs2429511 | 0.004266 | 0.84 | ADRB1 |
| rs5880 | 0.004652 | 2.41 | CETP |
| rs885834 | 0.010019 | 0.75 | CHAT |
| rs4802071 | 0.017725 | −0.73 | AKT2 |
| rs1951795 | 0.018991 | −1.18 | HIF1A |
| rs1478290 | 0.019807 | −0.76 | GYS2 |
| rs1042713 | 0.020062 | 1.03 | ADRB2 |
| rs2470890 | 0.028013 | −0.68 | CYP1A2 |
| rs1042718 | 0.028543 | −0.84 | ADRB2 |
| rs11212515 | 0.031215 | 0.67 | ACAT1 |
| rs2071710 | 0.032188 | −0.85 | SSTR3 |
| rs2301108 | 0.033986 | −1.14 | HIF1A |
| rs10841044 | 0.034345 | −1.10 | PIK3C2G |
| rs2241220 | 0.040482 | −1.11 | ACACB |
| rs6265 | 0.045395 | −0.74 | BDNF |
| rs10890819 | 0.049756 | 0.76 | ACAT1 |

Body Mass Index (BMI) Change

| | | | |
|---|---|---|---|
| rs814628 | 0.000517 | −1.17 | LIPF |
| rs4531 | 0.005455 | −0.87 | DBH |
| rs2306179 | 0.006943 | −0.60 | GYS2 |
| rs4987059 | 0.011513 | −1.05 | DRD4 |
| rs5883 | 0.014221 | −0.75 | CETP |
| rs5361 | 0.015981 | −0.57 | SELE |
| rs877172 | 0.025877 | 0.46 | OXT |
| rs1045642 | 0.036369 | −0.36 | ABCB1 |
| rs1128503 | 0.0389 | −0.45 | ABCB1 |
| rs4986894 | 0.039966 | −0.57 | CYP2C19 |
| rs2742115 | 0.040845 | 0.70 | OLR1 |
| rs694066 | 0.041227 | 0.63 | GAL |
| rs563895 | 0.041865 | 0.67 | AVEN |
| rs3750546 | 0.042248 | 0.45 | RXRA |
| rs3791850 | 0.042502 | −0.65 | GAD1 |
| rs2702285 | 0.043275 | 0.49 | AVEN |
| rs1290443 | 0.043733 | 0.46 | RARB |
| rs2033447 | 0.046514 | 0.38 | RARB |
| rs8178847 | 0.047652 | 0.65 | APOH |
| rs10422283 | 0.048702 | 0.47 | LIPE |

The SNPs and genes in Table 3 are provided in the nomenclature adopted by the National Center for Biotechnology Information (NCBI) of the National Institute of Health. The sequence data for the SNPs and genes listed in Table 3 is know in the art and is readily available from the NCBI dbSNP and OMIM databases. The coefficients are for the single SNPs and explain the residual change in the indicated response after covariates.

In another embodiment, the present invention provides a screening method to allow the identification of subsets of individuals who have specific genotypes and physiological characteristics and are more or less likely to respond favorably to VLC dieting. For example, a screening method of this embodiment involves obtaining a sample from an individual undergoing testing, such as a blood sample, and employing an assay method, e.g. the array system and newly-identified marker genes and gene variants as described, to evaluate whether the individual has a genotype associated with response to VLC dieting, and in particular change in blood LDL, HDL and triglyceride levels as well as change in body mass index. These individual's, who are identified through screening using the methods of the present invention, would be especially responsive to VLC dieting.

In another embodiment, a diagnostic system containing a support or support material, such as, without limitation, a nylon or nitrocellulose membrane, bead, or plastic film, or glass, or micro- or nano-array, comprising the novel set of genes as described herein, in a form suitable for the practitioner to employ in screening individuals. The diagnostic system can contain the novel gene marker set, or a subset of these genes, on a suitable substrate or micro- or nano-array. In addition, the diagnostic system can optionally contain other materials necessary for carrying out the assay method, including, but not limited to, labeled or unlabeled nucleic acid probes, detection label, buffers, controls, and instructions for use.

The following example demonstrates preferred embodiments of the invention. It should be appreciated by those of skill in the art that the techniques disclosed in the example which follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The content of all patents, patent applications, published articles, abstracts, books, reference manuals, sequence accession numbers, as cited herein are hereby incorporated by reference in their entireties to more fully describe the state of the art to which the invention pertains.

### EXAMPLE 1

### Physiogenomic Analysis of Patient Response to Interventional Low-Carbohydrate Dieting

Physiogenomics were used as a technique to explore the variability in patient response to VLC diet. Physiogenomics is a medical application of sensitivity analysis [30]. Sensitivity analysis is the study about the relations between the input and the output of a model and the analysis utilizing systems theory, of how variation of the input leads to changes in output quantities. Physiogenomics utilizes as input the variability in genes, measured by single nucleotide polymorphisms (SNP) and determines how the SNP frequency among individuals relates to the variability in physiological characteristics, the output.

The goal of the investigation was to develop physiogenomic markers for predicting anthropomorphic, lipid and endocrine effects of diets by using an informatics platform to analyze data from interventional dietary studies.

Materials and Methods

Patient enrollment. Overweight/obese men and women with a body mass index (BMI) 25 to 35 kg/m² and age 20 to 60 years were recruited. Initially, prospective volunteers were screened to obtain height, body weight, blood pressure, and medical, nutrition, and activity information. Subjects with Type I or II diabetes, liver or other metabolic/endocrine dysfunction, use of medications/supplements that affect cholesterol (e.g., statins, fish oil, nicotinic acid) or glucose (e.g., metformin), weight reducing or VLC diets, and blood pressure >160/95 mmHg were excluded. Previously active subjects were required to maintain their current exercise routines during the entire experimental period (verified by activity records) and sedentary individuals were not allowed to start an exercise program in order to control for possible confounding effects on the dependent variables [31].

Low attrition was credited to meticulous attention to dietary protocols with group and individualized counseling for subjects. Prospective volunteers were recruited from the local area around the University of Connecticut (Storrs, Conn.) including faculty, staff, graduate and undergraduate students at the main campus. Volunteer screening was performed without restriction in regard to gender, race, and socioeconomic status.

Diet Interventions. The interventions were VLC diets similar to diets investigated in several prior studies [23-29]. VLC diets have been studied extensively in terms of their effects on weight loss, body composition, blood lipids, and hormones and were selected over low-fat diets based on clinical work comparing VLC and low-fat diets [21, 23-29, 32-39].

The diet was hypocaloric (−500 kcal/day) based on estimated caloric needs to maintain body weight. A daily multi-vitamin/mineral complex at levels ≦100% of the RDA was given to subjects to ensure adequate micronutrient status. Subjects were required to report to the laboratory each week for group meetings that involved education and assessment of weight, dietary compliance, and ketone monitoring.

The main goal of the VLC diet was to restrict carbohydrate to <10% of total energy. Customized diabetic exchange lists were used to ensure a constant energy and balance of protein (˜25% of energy), fat (˜65% of energy), and carbohydrate (˜10% of energy) throughout the day. There were no restrictions on the type of fat from saturated and unsaturated sources or cholesterol levels. Foods commonly consumed included beef (e.g., hamburger, steak), poultry (e.g., chicken, turkey), fish, oils, nuts/seeds, peanut butter, moderate amounts of vegetables, salads with low-carbohydrate dressing, moderate amounts of cheese, eggs, protein powder, and water or low-carbohydrate diet drinks. Excellent compliance was attained as measured by production of urinary and blood ketones, indicating a high degree of dietary compliance in terms of carbohydrate restriction. Subjects tested and recorded their urine ketones daily using reagent strips (Bayer Corporation, Elkhart, Ind.).

Body Mass and Body Composition. Body mass and body composition were measured in the morning after an overnight fast. Body mass was recorded to the nearest 100 g on a calibrated digital scale with subjects either nude or wearing only underwear. Whole body and regional body composition were assessed using a state-of-the-art fan-beam DXA (Prodigy™, Lunar Corporation, Madison, Wis.). Analyses were performed by the same blinded technician. Regional analysis of the abdomen was assessed by placing a box between L1 and L4 using commercial software (enCORE version 6.00.270). This abdominal region of interest has been shown to be a highly reliable and accurate determinant of abdominal obesity compared to multi-slice computed tomography [40]. It has previously been found that, compared to a LF diet, a VLC diet results in preferential loss of fat in this abdominal region [25].

Fasting Blood Collection. Blood lipids, insulin and glucose were determined by standard methods reported by Volek et al. [26].

Clinical Database. As a result of the above measurements in the recruited subjects, a clinical database was created. This work has generally shown that VLC diets have a favorable effect on biomarkers for cardiovascular disease. A VLC diet reduces fasting triacylglycerols, increases HDL cholesterol, and promotes a more favorable LDL subclass pattern. Furthermore, a VLC diet results in significant reductions in the triacylglycerol response to a fat-rich meal (i.e., decreased postprandial lipemia).

A great deal of variability in response to diet is evident among subjects. For example, individual variability in LDL-C, HDL-C and triglycerides responses to a VLC diet were been studied. For LDL-C, the response was evenly split (half increase, half decrease) such that the mean response was zero. For HDL-C, there was an average improvement, but with significant variability. For triglycerides, almost all subjects exhibited a decrease but there was large variability in the magnitude of the reduction. Similar responses showing variability exist for other parameters such as weight loss, fat loss, insulin, and LDL size in response to VLC and LF diets. These results show the variability in response to the same diet and provide strong evidence for the need to individualize the diet prescription. Of note is the fact that two components of metabolic syndrome, HDL and TG respond favorably on average to carbohydrate-restricted diets.

A basic question pertains to which particular variables might explain this variability in response. To this end, the effect of various baseline covariates on dietary response were examined. Baseline LDL, HDL and TG, weight, percentage body fat and gender were determined. These covariates explain 30% of the variability in LDL response, and 50% of the variability in HDL and TG response. To improve the precision of the prediction, it was hypothesized that a unique combination of genetic, physiological, and demographic information (a PhysioType™) can precisely predict the response.

Sample procurement and DNA isolation. DNA samples were quantified by the fluorescent PicoGreen assay and diluted or concentrated to a standard 50 ng/uL. Each DNA sample was examined according to a standard PCR analytical panel to determine its suitability for the Illumina BeadStation. It has been observed that in many clinical studies the range of DNA quality, concentration and suitability for amplification is quite variable and reflects legacy issues related to whole blood extraction and banking over several studies. For example, depending on the extraction technique, hemoglobin remnants and heme itself may interfere with DNA amplification by inhibition of the DNA polymerase. The Whole Genome Amplification (WGA) technology is particularly useful in this study. WGA allows trace DNA remnants in blood serum to be amplified and provide DNA template for subsequent PCR analysis via multiple displacement amplification [41]. WGA has successfully been used to amplify microgram amounts of genomic DNA (gDNA) from the low number of gDNA copies present in serum. Genomic DNA was isolated from whole blood using standard DNA extraction and purification methods and reagents (Qiagen, San Diego, Calif.). WGA was performed on stored and cryo-preserved plasma samples where whole blood could not be obtained. The quality of DNA was determined by amplification of two loci highly sensitive to degradation using TaqMan (Applied Biosystems) [42]. The concentration of double stranded DNA was determined using the PicoGreen assay (Molecular Probes, Eugene, Oreg.). gDNA was adjusted to 50 ng/μL, aliquoted into 96-well plates and stored at −86° C.

Development of Nutrition Gene Array

In order to obtain multiple genotypes for the recruited subjects in the diet study, a Nutrition Gene Array was be developed, consisting of nutritionally-relevant genetic markers associated with lipid metabolism, metabolic/endocrine function, vascular inflammation endothelial dysfunction and obesity.

Selection of Gene Markers. To determine the influence of genetic factors on VLC dieting, genes associated with dyslipidemia, metabolic and endocrine function, endothelial dysfunction, and chronic vascular inflammation provided the initial focus. Additional genes relevant to nutrition were also included. The physiogenomic rationale for the selection of these genes is as follows. First, these genes are representatives of the various physiological pathways and networks. The use of these genes in physiogenomics is in sharp contrast to gene discovery efforts based on gene expression profiling or disease mapping. It will be recognized that that this list of genes will miss some known key genes, and will certainly lack those genes not discovered so far or not identified yet as relevant. Second, as many of the physiological networks have built in redundancy, feedback, and amplification, it is assumed that the elucidation of every single gene in a pathway may be unnecessary for physiogenomics as long as a representative gene of a given circuit is included.

Physiogenomics posits the gene representation question in the following logic: representative genes are selected based on various functional criteria, the genes are assembled in a panel, and the panel is then used as a substrate to draw inferences on physiological association based on genetics. Through clinical research the predictive power of the panel is ascertained. The underlying hypothesis is that the genes in the panel together explain a useful fraction of the variability in response among individuals. If the answer is affirmative, the hypothesis is accepted, and the panel is used. If the answer is inconclusive, the roster of genes is modified until the panel's predictive level is clinically useful.

The combination of several disease factors will lead to insulin resistance, MetSyn, atherosclerosis, hypertension, thrombosis and eventually cardiovascular events. Many of the selected genes have functions in more than just one category. The selection criteria considered each gene's known physiological and pathophysiological importance in respect to the MetSyn and CVD [43]. The existence of functionally active mutations (OMIM Database, 2000) [44] was considered but not required for selection. A subset of the selected genes has functional genotypes that represent risk factors for the development of atherosclerosis (e.g., apoE), hypertension (e.g., angiotensinogen), obesity (e.g., leptin), or insulin resistance (e.g., insulin receptor). It was expected that a stronger correlation between the risk of an individual to develop CVD and the benefits of diet would be obtained when using an array of genes as markers compared to a small set of clinical markers, such as lipid profiles. The genes of the Nutrition Array are discussed below.

a. Lipid Metabolism and Dyslipidemia. Along with their essential role in energy homeostasis, organ physiology, and cellular biology, lipids are linked to many pathological processes. Lipoproteins function as carrier molecules for lipids, cholesterol, and cholesterol esters. Apolipoproteins, the structural components of these lipoprotein transport molecules, are being studied in CVD for their role in atherosclerotic plaques development [45]. They assist in the transport of cholesterol from bodily tissues to the liver for excretion (APOA1) and in the transport and conversion of TGs (APOB). Apoliporoteins are also involved in the metabolism of TG-rich lipoproteins (APOE) and represent cofactors for lipid modifying proteins (APOA1 for lecithin:cholesterol acyltransferase {LCAT}, APOC for lipoprotein lipase {LPL}). Enzymes participating in cholesterol synthesis, uptake and modification represent valuable targets for an anti-atherosclerotic approach, documented by the current development of new lipid-lowering agents, such as inhibitors of CETP and ACAT1 and PPAR agonists [46]. LPL plays an important role in VLDL fatty acid release and its subsequent conversion to LDL. Hormone-sensitive lipase (HSL) is a major determinant of fatty acid mobilization. It plays a pivotal role in lipid metabolism, overall energy homeostasis, and fatty acid signaling. Two proteins have been selected as part of the free fatty acid metabolisms [47]. Carnitine palmitoyltransferase (CPT) facilitates mitochondrial fatty acid oxidation and deficiencies in CPT are common disorders. The intestinal fatty acid binding protein (FABP2) gene is of interest since it has been proposed as a candidate gene for diabetes. Cells are endowed with 2 acetyl-coenzyme A carboxylase (ACC) systems to control fatty acid amounts and ACCB is believed to control mitochondrial fatty acid oxidation.

b. Metabolic or Endocrine Function. Human evolution selected genes that mediate the efficient conversion of nutrients into fat as an effective storage form. Ingestion of a high carbohydrate diet results in increased insulin levels. Among the regulatory enzymes of glycolysis and lipogenesis that become activated [48], pyruvate kinase (PK), phosphofructokinase (PFK), acetyl CoA carboxylase (ACC), and fatty acid synthase (FAS) were selected for genomic analysis. The transcriptional regulation of those genes is facilitated by the carbohydrate response element-binding protein (CHREBP) [49]. Similarly, insulin stimulated lipogenesis is mediated through a transcription factor called sterol responsive element-binding protein (SREBP). It controls genes involved in cholesterol uptake and biosynthesis. ATP-binding cassette (ABC) transporters modulate cholesterol and lipoprotein metabolism [45]. ABCG5 and ABCC8 play an important role in limiting intestinal absorption and promoting biliary excretion of neutral sterols. PPARs are members of the nuclear receptor superfamily [50]. Two members, PPARA and PPARG, regulate fatty acid catabolism, adipocyte differentiation, lipid storage, and glucose homeostasis. PPAR agonists have all been reported to exhibit anti-inflammatory activity in macrophages and endothelial cells. Glycogen synthase (GYS) activity is thought to be rate-limiting in the disposal of glucose as muscle glycogen [51]. Phosphoenolpyruvate carboxykinase (PEPCK) is considered to be the first step in gluconeogensis. The synthesis of the soluble isoform (PEPCK1) is regulated by gene transcription and the rate of mRNA turnover can be induced by starvation and reduced through a high carbohydrate diet. Adiponectin and resistin are secretory products of adipose tissue [52]. Plasma adiponectin is reduced in MetSyn and in patients with ischemic heart disease. Hypoadiponectinemia may contribute to insulin resistance and accelerated atherogenesis in obesity. UCP2 and UCP3 play a role in reducing reactive oxygen species formation. UCP3 could also facilitate lipid oxidation by acting as a free fatty acid anion transporter in a variety of physiological states. UCP3 represents an interesting target shifting energy expenditure towards heat dissipation.

c. Vascular Inflammation. In recent years, it has become apparent that low-grade vascular inflammation plays a key role in all stages of the atherosclerotic process [53]. Several blood markers indicative of endothelial dysfunction and vascular inflammation have been found to be associated with future cardiovascular risk including proinflammatory cytokines, such as IL-6 and TNF-α and the acute phase reactant CRP [54]. During early atherosclerotic lesion development the activated endothelium releases cellular adhesion molecules [53] that lead to the adherence and transendothelial migration of monocytes (through P-selectin and E-selectin) and leukocytes (through ICAM1 and VCAM1). Inflammatory cytokines such as IL-1, TNF-α, interferon-γ, or oxidized LDL receptor modulate the expression of E-selectin, ICAM1, and VCAM1. Cytokine stimulated endothelial cells also produce MCP-1 and IL-6, which further amplify the inflammatory cascade. TNF-α is produced by a variety of cells. TNF-α stimulates, along with interferon-γ and IL-1, the production of IL-6 by smooth muscle cells. IL-6 gene transcripts are expressed in human atheromatous lesions, and IL-6 is the main hepatic stimulus for CRP production. CRP may contribute directly to the proinflammatory state by stimulating the release of inflammatory cytokines such as IL-1β, IL-6, and TNF-α by endothelial cells.

d. Endothelial Dysfunction. The endothelium regulates vascular tone through the release of vasoactive substances [55]. The two most important vasocontrictors are endothelin and angiotensin II. Angiotensin II stimulates a variety of pro-atherogenic responses, such as expression of adhesion molecules (e.g., ICAM1, VCAM1), platelet aggregation, thrombosis (through PAI1 expression), cell migration, and expression of TGF-1β. We are therefore interested in genetic modifications of the AGT, the precursor of angiotensin II. AGT is processed by the renin-angiotensin (ACE) system. The most important vasodilator is NO, generated by the endothelial nitric oxide synthase (NOS3) [56]. NO is also vascular protective and inhibits inflammation, oxidation, vascular smooth muscle cell proliferation, and migration. Endothelial dysfunction is caused by a damaged endothelium with impaired NO release. Reduction in bioavailable NO can be a result of altered NOS3 expression or activity, but is often due to a decrease in NO half-life. The reaction of NO with superoxide is extremely fast and efficient. Superoxide dismutase protects NO and endothelial function [57]. The link between endothelial dysfunction and traditional risk factors for CVD, including diabetes, hypercholesterolemia, and hypertension, supports the effort to include related genes [58].

e. Obesity. Genes involved in the regulation of energy metabolism, appetite control or autocrine-paracrine signalling by adipocytes are all plausible candidates for genes that are involved in common obesity. Adiponectin is a hormone that regulates energy homeostasis and glucose and lipid metabolism. It is expressed by differentiated adipocytes as a 33-kD protein that is also detectable in serum [59]. Leptin is a protein that plays a critical role in the regulation of body weight by inhibiting food intake and stimulating energy expenditure. Multiple regression analysis has shown that adiposity, gender, and insulinemia were significant determinants of leptin concentration, explaining 42%, 28%, and 2% of its variance, respectively [60]. Uncoupling protein-1 (UCP-1) diverts energy from ATP synthesis to thermogenesis in the mitochondria of brown adipose by catalyzing a regulated leak of protons across the inner membrane. Manipulation of thermogenesis could be an effective strategy against obesity [61]. The solute carrier family 6 member 14 gene (SLC6A14) gene encodes a sodium- and chloride-dependent transporter of neutral and cationic amino acids [62] that has a high affinity for the non-polar amino acid tryptophan. In the brain, the enzyme tryptophan hydroxylase converts tryptophan into serotonin. This neurotransmitter is known to be strongly involved with the central signaling of satiety by mechanisms that include effects on downstream effector neurons in the hypothalamus [63]. Therefore, a possible hypothesis is that a reduction in the concentration of serotonin owing to reduced tryptophan transport might be the link with lower SLC6A14 activity, and thereby increasing susceptibility to obesity by reducing satiety [64].

Potential associations to diet using the Nutrition Gene array. Various SNPs associated with the observation of lipid level and BMI changes in patients undergoing a low-carbohydrate diet were screened. The endpoints analyzed were the blood levels of LDL, HDL and triglycerides (TG) and BMI. The physiogenomic model was developed using the following procedure: 1) Establish a baseline model using only the demographic and clinical variables, 2) Screen for associated genetic markers by testing each SNP against the unexplained residual of the baseline model, and 3) Establish a revised model incorporating the significant associations from the SNP screen. All models are simple linear regression models, but other well-known statistical methods are contemplated to be useful.

The means of the baseline variables broken down by demographic factors are shown in Table 4.

TABLE 4

| Factor* | value | N | TC | LDL | HDL | TG | logTG | THR | BMS | FMS | LBM | % Fat | BMI |
|---|---|---|---|---|---|---|---|---|---|---|---|---|---|
| Gender | female | 33 | 186.0 | 111.3 | 46.0 | 93.0 | 4.5 | 4.0 | 72.0 | 30.8 | 41.6 | 41.3 | 27.7 |
| Gender | male | 53 | 183.5 | 114.0 | 38.0 | 129.0 | 4.9 | 4.9 | 96.8 | 31.7 | 61.3 | 33.1 | 30.7 |
| Age | <40 | 56 | 182.9 | 110.6 | 38.2 | 123.8 | 4.8 | 4.5 | 88.6 | 31.8 | 51.1 | 34.9 | 29.7 |
| Age | 40-49 | 21 | 181.5 | 114.5 | 43.0 | 116.6 | 4.8 | 4.5 | 95.5 | 30.8 | 60.2 | 33.2 | 30.5 |
| Age | 50-59 | 6 | 202.8 | 142.5 | 39.5 | 126.2 | 4.8 | 5.3 | 96.3 | 32.3 | 59.2 | 33.1 | 29.1 |
| Age | 60-69 | 3 | 162.4 | 92.6 | 48.0 | 65.5 | 4.2 | 3.3 | 83.0 | 23.5 | 56.8 | 28.1 | 26.9 |
| Ethnicity | AfricanAm | 5 | 161.0 | 100.4 | 38.0 | 93.0 | 4.5 | 4.1 | 68.8 | 27.2 | 37.0 | 34.7 | 27.7 |
| Ethnicity | Asian | 1 | 161.0 | 111.3 | 31.5 | 91.0 | 4.5 | 5.1 | 60.9 | 22.4 | 36.4 | 36.5 | 24.1 |
| Ethnicity | Caucasian | 74 | 186.5 | 114.3 | 40.0 | 120.0 | 4.8 | 4.5 | 92.4 | 31.8 | 56.8 | 34.1 | 30.3 |
| Ethnicity | Hispanic | 3 | 188.5 | 108.5 | 36.5 | 168.9 | 5.1 | 5.3 | 86.7 | 25.9 | 39.4 | 27.2 | 27.7 |
| Ethnicity | India | 1 | 203.8 | 135.6 | 42.0 | 131.1 | 4.9 | 4.9 | 73.8 | 25.1 | 46.3 | 33.8 | 25.9 |
| Ethnicity | Indian | 2 | 160.4 | 104.3 | 34.9 | 106.4 | 4.6 | 4.6 | 89.5 | 29.9 | 56.7 | 32.7 | 28.7 |
| Order | first | 67 | 188.0 | 115.0 | 40.0 | 129.0 | 4.9 | 4.8 | 92.3 | 32.1 | 56.7 | 34.8 | 30.2 |
| Order | second | 19 | 167.0 | 111.0 | 45.0 | 85.0 | 4.4 | 3.8 | 72.0 | 25.3 | 42.5 | 32.8 | 26.9 |
| *Gender (Male, female); Age; Ethnicity (Self described); Order (First, second. If second, patient was on a low fat diet previously); Length (Number of weeks of diet); Tc.pre (Total cholesterol (mg/dL)); Ldl.pre (LDL cholesterol (mg/dL)); Hdl.pre (HDL cholesterol (mg/dL)); | | | | | | | | | | | | | |
| # Tg.pre (Triglycerides (mg/dL)); Logtg.pre (Natural logarithm of Tg); Thr.pre (Ratio between TC and HDL); Bms.pre (Body mass (kg)); Fms.pre (Fat mass (kg)); Lbm.pre (Lean body mass (kg)); Pcfat.pre (Percentage of total body mass that is fat); Bmi.pre (Body mass index). | | | | | | | | | | | | | |

In the SNP screen (step 2), the p-values for each SNP were obtained by adding the SNP to the baseline model and comparing the resulting model improvement with up to 10,000 simulated model improvements using the same data set, but with the genotype data randomly permuted to remove any true association. This method produces a p-value that is a direct, unbiased, and model-free estimate of the probability of finding a model as good as the one tested when the null hypothesis of no association is true. All SNPs with a screening p-value of better than 0.003 were selected to be included in the physiogenomic model (step 3).

Data Analysis. Covariates were analyzed using multiple linear regression and the stepwise procedure. An extended linear model was constructed including the significant covariate and the SNP genotype. SNP genotype was coded quantitatively as a numerical variable indicating the number of minor alleles: 0 for major homozygotes, 1 for heterozygotes, and 2 for minor homozygotes. The F-statistic p-value for the SNP variable was used to evaluate the significance of association. Table 3 lists all SNPs that were tested and their association p-values. The validity of the p-values were tested by performance of an independent calculation of the p-values using permutation testing. To account for the multiple testing of multiple SNPs, adjusted p-values were calculated using Benjamini and Hochbergs false discovery rate (FDR) procedure [65,66,67]. In addition, the power for detecting an association based on the Bonferroni multiple comparison adjustment was evaluated. For each SNP, the effect size in standard deviations that was necessary for detection of an association at a power of 80% (20% false negative rate) was calculated using the formula:

$$Δ = \frac{{z}_{α / c} + {z}_{β}}{\sqrt{Nf ( 1 - f )}}$$

where α was the desired false positive rate (α=0.05), β the false negative rate (β=1-Power=0.2), c the number of SNPs, z a standard normal deviate, N the number of subjects, f the carrier proportion, and Δ the difference in change in response between carriers and non-carriers expressed relative to the standard deviation [68].

LOESS representation. A locally smoothed function of the SNP frequency as it varies with each response was used to visually represent the nature of an association. LOESS (LOcally wEighted Scatter plot Smooth) is a method to smooth data using a locally weighted linear regression [69, 70]. At each point in the LOESS curve, a quadratic polynomial was fitted to the data in the vicinity of that point. The data were weighted such that they contributed less if they were further away, according to the following tricubic function where x was the abscissa of the point to be estimated, the xᵢ were the data points in the vicinity, and d(x) was the maximum distance of x to the xᵢ.

$${w}_{i} = {( 1 - {\vert \frac{x - {x}_{i}}{d ( x )} \vert}^{3} )}^{3}$$

### Results

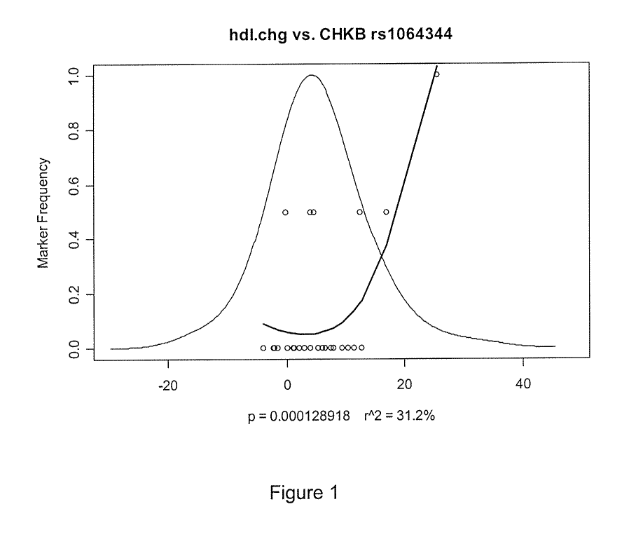

The distribution of change in HDL values in the study population was approximately normal (FIG. 1). The potential covariates of age, gender, race, were tested for association with HDL change using multiple linear regression.

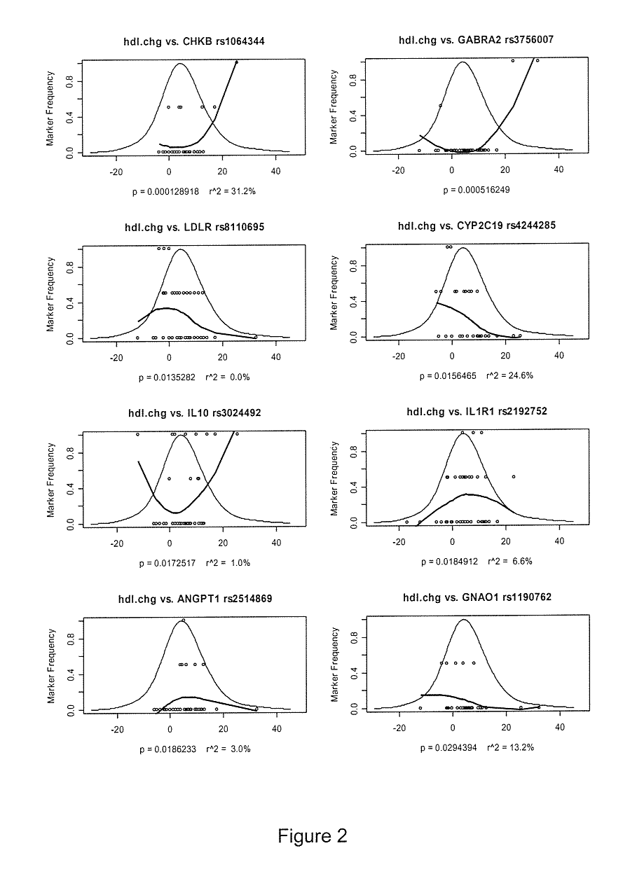

The overall distribution of change in HDL values is shown in FIG. 2 along with the individual genotypes and a LOESS fit of the allele frequency as a function of change in HDL values. The bell curve shows the actual distribution of HDL phenotype in the clinical database. The LOESS curve shows the localized frequency of the least common allele for sectors of the distribution. For SNPs with a strong association, the marker frequency is significantly different between the high end and the low end of the distribution. Conversely, if a marker is neutral, the frequency is independent of the response and the LOESS curve is essentially flat.

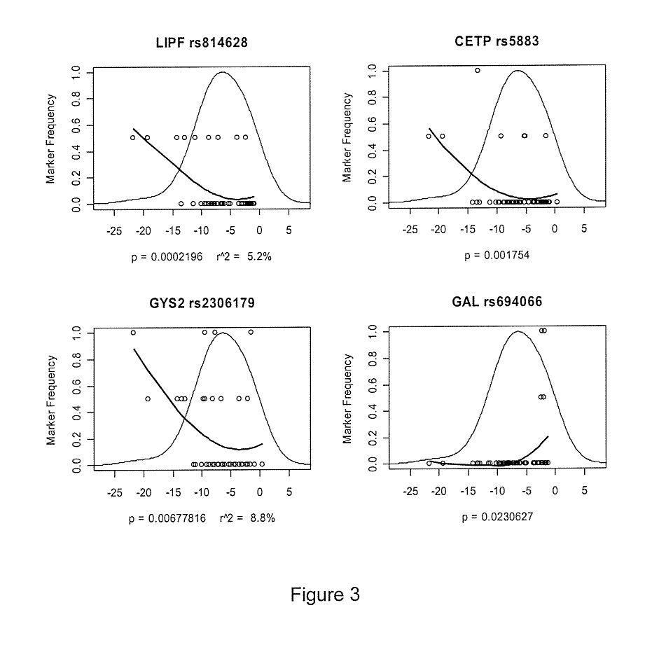

FIG. 3 shows LOESS plots for four SNPs as a function of change in BMS. The thin line outlines the overall distribution of the response variable. The mean is around −6, indicating that, on average, each individual lost 6 kg during the diet. The dots indicate genotype and phenotype for each individual. The bottom row (frequency=0) are the patients with the common variant (major allele), the middle row (frequency=0.5) are the heterozygotes, the top row are the homozygotes for the rare variant (minor allele). The x-position of the dot indicates the patient's weight loss in response to the diet. The thick line is a smooth fit indicating the estimated frequency of the allele in a slice of the population experiencing a particular weight loss response. If the allele is more common among patients with high weight loss than among those with low weight loss, the allele is likely to be associated with increased response. Similarly, when the allele is less common in those with higher weight loss, the allele is associated with decreased response. Thus, the slope of the curve is an indication of the degree of association.

### Statistical Plan

a. Data analysis The objective of the statistical analysis is to find a set of physiogenomic factors that together provide a way of predicting the outcome of interest. The association of an individual factor with the outcome may not have sufficient discrimination ability to provide the necessary sensitivity and specificity, but by combining the effect of several such factors the objective is reached. Increased sensitivity and specificity for the cumulative effect on prediction can be achieved through the use of common factors that are statistically independent. The assumptions on which these calculations are based are (a) the factors are independent of each other, (b) the association between each factor and the outcome can be summarized by a modest odds ratio of 1.7, and (c) the prevalence of each physiogenomic factor in the population is 50% and independent of the others. Clearly, the prediction becomes even stronger if the association with the response is stronger or one finds additional predictors. However, factors that are less useful for these types of prediction are those that are less common in the population, or collinear with factors that have already been identified in the prediction model.

b. Model Building. Discovery of markers affecting response to diet. A model was developed for the purpose of predicting a given response (Y) to a diet; including change in anthropomorphic, lipid, inflammatory, endothelial and endocrine effects. A linear model for subjects in the diet group was be used in which the response of interest can be expressed as follows:

$$Y = {R}_{0} + {\sum }_{i} {α}_{i} {M}_{i} + {\sum }_{j} {β}_{j} {D}_{j} + ɛ$$

where Mᵢ are the dummy marker variables indicating the presence of specified genotypes and Dⱼ are demographic and clinical covariates. The model parameters that are to be estimated from the data are R₀, αᵢ and βⱼ. This model employs standard regression techniques that enable the systematic search for the best predictors. S-plus provides very good support for algorithms that provide these estimates for the initial linear regression models, as well other generalized linear models that may be used when the error distribution is not normal. For continuous variables, generalized additive models, including cubic splines in order to appropriately assess the form for the dose-response relationship may also be considered [71,72].

In addition to optimizing the parameters, model refinement is performed. The first phase of the regression analysis will consist of considering a set of simplified models by eliminating each variable in turn and re-optimizing the likelihood function. The ratio between the two maximum likelihoods of the original vs. the simplified model then provides a significance measure for the contribution of each variable to the model.

The association between each physiogenomic factor and the outcome is calculated using logistic regression models, controlling for the other factors that have been found to be relevant. The magnitude of these associations are measured with the odds ratio and the corresponding 95% confidence interval, and statistical significance assessed using a likelihood ratio test. Multivariate analyses is used which includes all factors that have been found to be important based on univariate analyses.

Because the number of possible comparisons can become very large in analyses that evaluate the combined effects of two or more genes, the results include a random permutation test for the null hypothesis of no effect for two through five combinations of genes. This is accomplished by randomly assigning the outcome to each individual in the study, which is implied by the null distribution of no genetic effect, and estimating the test statistic that corresponds to the null hypothesis of the gene combination effect. Repeating this process 1000 times will provide an empirical estimate of the distribution for the test statistic, and hence a p-value that takes into account the process that gave rise to the multiple comparisons. In addition, hierarchical regression analysis is considered to generate estimates incorporating prior information about the biological activity of the gene variants. In this type of analysis, multiple genotypes and other risk factors can be considered simultaneously as a set, and estimates will be adjusted based on prior information and the observed covariance, theoretically improving the accuracy and precision of effect estimates [73].

c. Power calculations. The data available for study in this project are for 86 subjects. The power available for detecting an odds ratio (OR) of a specified size for a particular allele was determined on the basis of a significance test on the corresponding difference in proportions using a 5% level of significance. The approach for calculating power involved the adaptation of the method given by Rosner [68]. The SNPs that are explored in this research are not so common as to have prevalence of more than 35%, but rather in the range of 10-15%. Therefore, it is apparent that the study has at least 80% power to detect odds ratios in the range of 1.6-1.8, which are modest effects.

d. Model validation. A cross-validation approach is used to evaluate the performance of models by separating the data used for parameterization (training set) from the data used for testing (test set). The approach randomly divides the population into the training set, which will comprise 80% of the subjects, and the remaining 20% will be the test set. The algorithmic approach is used for finding a model that can be used for prediction of dietary response will occur in a subject using the data in the training set. This prediction equation is then used to prepare an ROC curve that provides an independent estimate of the relationship between sensitivity and specificity for the prediction model.

e. Patient Physiotype. Table 5 shows a collection of physiotypes for the outcomes LDL, HDL, TG, and BMI. Each physiotype in this particular embodiment consists of a selection of markers, and intercept value, and a coefficient for each marker. For example, the LDL physiotype consists of the marker rs1018381 and rs4804103, and the coefficients −1.28 and −0.83, respectively. The predicted LDL response for a given individual is then given by the formula

$$Δ LDL = C + {\sum }_{i} {c}_{i} {g}_{i}$$

where C is the intercept, the cᵢ are the coefficients and the gᵢ are the genotypes, coded 0 for the wild type allele homozygote, 1 for the heterozygote, and 2 for the variant allele homozygote as listed for an example individual in the DNA type column. For example, the LDL response for the individual specified in Table 5 would be predicted as −7.48, since the genotypes for both markers are zero and the intercept is −7.48.

In this embodiment, the physiotype consists of a linear regression model. In other embodiments, the physiotype might consist of a generalized linear regression model, a structural equation model, a Baysian probability network, or any other modeling tool known to the practitioner of the art of statistics.

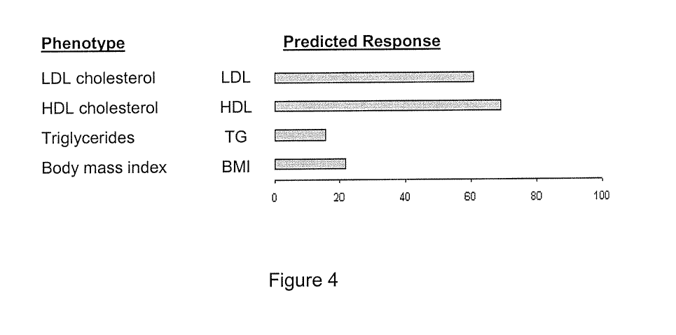

The patient's physiotype may be expressed in a convenient format for the practitioner's assessment of a patient's likely response to diet. The patient's physiotype corresponding to the genotype of Table 5 is shown in FIG. 4.

TABLE 5

| | | DNA | | | | | |
|---|---|---|---|---|---|---|---|
| Snp | Gene | Type | Alleles | LDL | HDL | TG | BMI |
| Intercept | | | | −7.48 | −3.43 | −5.05 | −0.24 |
| rs1018381 | DTNBP1 | 0 | CC | −1.28 | | | |
| rs4804103 | INSR | 0 | CC | −0.83 | | | |
| rs1064344 | CHKB | 0 | GG | | −0.23 | | |
| rs3756007 | GABRA2 | 1 | TC | | −0.46 | | |
| rs8110695 | LDLR | 0 | TT | | 0.10 | | |
| rs4244285 | CYP2C19 | 0 | GG | | 0.22 | | |
| rs3024492 | IL10 | 1 | AT | | −0.04 | | |
| rs2192752 | IL1R1 | 1 | AC | | −0.32 | | |
| rs2514869 | ANGPT1 | 0 | TT | | −0.36 | | |
| rs1190762 | GNAO1 | 0 | CC | | 0.54 | | |
| rs132642 | APOL3 | 0 | TT | | | −0.40 | |
| rs3757868 | ACHE | 0 | GG | | | −0.30 | |
| rs1951795 | HIF1A | 0 | CC | | | 0.59 | |
| rs3791981 | APOB | 0 | AA | | | −1.56 | |
| rs10460960 | LOC391530 | 0 | AA | | | 0.73 | |
| rs814628 | LIPF | 1 | AG | | | | 0.76 |
| rs4531 | DBH | 0 | GG | | | | −0.22 |
| rs2306179 | GYS2 | 1 | AG | | | | 0.56 |
| rs4987059 | DRD4 | 0 | GG | | | | 0.003 |
| rs5883 | CETP | 0 | CC | | | | 0.41 |
| rs5361 | SELE | 1 | AC | | | | −0.01 |
| rs877172 | OXT | 1 | AC | | | | −0.33 |

### REFERENCES

[1] Centers for Disease Control and Prevention's (CDC) National Center for Health Statistics, Health, United States, 2005.
[2] World Cancer Research Fund and American Institute for Cancer Research Food, Nutrition and the Prevention of Cancer: A Global Perspective. American Institute for Cancer Research, Washington, D.C. 1997.
[3] Wylie-Rosett J., Herman W. H., and Goldberg R. B., Curr. Opin. Lipidol., February 2006;17(1):37-44.
[4] ATP III (Adult Treatment Panel III), National Cholesterol Education Program. Detection, Evaluation, and Treatment of High Blood Cholesterol in Adults, Final Report. NIH Pub. No. 02-5215. 2002.
[5] Parks E J, Hellerstein M K. Carbohydrate-induced hypertriacylglycerolemia: historical perspective and review of biological mechanisms. Am J Clin Nutr 2000;71:412-33.
[6] Ginsberg H N, Kris-Etherton P, Dennis B, et al. Effects of reducing dietary saturated fatty acids on plasma lipids and lipoproteins in healthy subjects: the DELTA Study, protocol 1. Arterioscler Thromb Vasc Biol 1998;18:441-9.
[7] Yu-Poth S, Zhao G, Etherton T, Naglak M, Jonnalagadda S, Kris-Etherton P M. Effects of the National Cholesterol Education Program's Step I and Step II dietary intervention programs on cardiovascular disease risk factors: a meta-analysis. Am J Clin Nutr 1999;69:632-46.
[8] Retzlaff B M, Walden C E, Dowdy A A, McCann B S, Anderson K V, Knopp R H. Changes in plasma triacylglycerol concentrations among free-living hyperlipidemic men adopting different carbohydrate intakes over 2 y: the Dietary Alternatives Study. Am J Clin Nutr 1995;62:988-95.
[9] Berglund L, Oliver E H, Fontanez N, et al. HDL-subpopulation patterns in response to reductions in dietary total and saturated fat intakes in healthy subjects. Am J Clin Nutr 1999;70:992-1000.
[10] Dreon D M, Fernstrom H A, Miller B, Krauss R M. Low-density lipoprotein subclass patterns and lipoprotein response to a reduced-fat diet in men. Faseb J 1994;8:121-6.
[11] Dreon D M, Fernstrom H A, Williams P T, Krauss R M. A very low-fat diet is not associated with improved lipoprotein profiles in men with a predominance of large, low-density lipoproteins. Am J Clin Nutr 1999;69:411-8.
[12] Blackburn, G. L. Phillips, J. C., Morreale, S., Physician's guide to popular low-carbohydrate weight-loss diets. Cleve. Clin. J. Med. 2001; 68:761-74.
[13] Volek, J. S. and Sharman, M. J., Cardiovascular and Hormonal Aspects of Very-Low-Carbohydrate Ketogenic Diets. Obesity Research, Vol. 12 Supplement November 2004, pp 115s-123s.
[14] Ordovas J M. Gene-diet interaction and plasma lipid responses to dietary intervention. Biochem Soc Trans 2002;30:68-73.
[15] Ordovas J M, Corella D, Cupples L A, et al. Polyunsaturated fatty acids modulate the effects of the APOA1 G-A polymorphism on HDL-cholesterol concentrations in a sex-specific manner: the Framingham Study. Am J Clin Nutr 2002a;75:38-46.
[16] Ordovas J M, Corella D, Demissie S, et al. Dietary fat intake determines the effect of a common polymorphism in the hepatic lipase gene promoter on high-density lipoprotein metabolism: evidence of a strong dose effect in this gene-nutrient interaction in the Framingham Study. Circulation 2002b;106:2315-21.
[17] Arab L. Individualized nutritional recommendations: do we have the measurements needed to assess risk and make dietary recommendations? Proc Nutr Soc 2004;63:167-72.
[18] Chadwick R. Nutrigenomics, individualism and public health. Proc Nutr Soc 2004;63:161-6.
[19] German J B, Roberts M A, Watkins S M. Personal metabolomics as a next generation nutritional assessment. J Nutr 2003;133:4260-6.
[20] Gillies P J. Nutrigenomics: the Rubicon of molecular nutrition. J Am Diet Assoc 2003;103:S50-5.
[21] Volek, J. S., A. L. Gómez, and W. J. Kraemer (2000): Fasting and postprandial lipoprotein responses to a low-carbohydrate diet supplemented with n-3 fatty acids. J. Am. Coll. Nutri. 19: 383-391.
[22] Volek, J. S., A. L. Gómez, D. M. Love, N. G. Avery, M. J. Sharman, and W. J. Kraemer (2001): Effects of a high-fat diet on postabsorptive and postprandial testosterone responses to a fat-rich meal. Metabolism: Clin. Exp. 50: 1351-1355, 2001.
[23] Volek, J. S., M. J. Sharman, D. M. Love, N. G. Avery, A. L. Gómez, T. P. Scheett, and W. J. Kraemer (2002): Body composition and hormonal responses to a carbohydrate-restricted diet. Metabolism: Clin. Exp. 51: 864-70.
[24] Volek, J. S., M. J. Sharman, A. L. Gómez, T. P. Scheett, and W. J. Kraemer (2003). An isoenergetic very low-carbohydrate diet is associated with improved serum high-density lipoprotein cholesterol (HDL-C), total cholesterol to HDL-C ratio, triacylglycerols, and postprandial lipemic responses compared to a low-fat diet in normal weight, normolipidemic women. J. Nutr. 133: 2756-2761.
[25] Volek J S, Sharman M J, Gomez A L, et al. Comparison of energy-restricted very low-carbohydrate and low-fat diets on weight loss and body composition in overweight men and women. Nutr Metab (Lond) 2004a;1:13.
[26] Volek J S, Sharman M J, Gomez A L, et al. Comparison of a very low-carbohydrate and low-fat diet on fasting lipids, LDL subclasses, insulin resistance, and postprandial lipemic responses in overweight women. J Am Coll Nutr 2004b;23:177-84.
[27] Sharman M J, Volek J S. Weight loss leads to reductions in inflammatory biomarkers after a very-low-carbohydrate diet and a low-fat diet in overweight men. Clin Sci (Lond) 2004;107:365-9.
[28] Sharman, M. J. W. J. Kraemer, D. M. Love, N. G. Avery, A. L. Gómez, T. P. Scheett, and J. S. Volek (2002): A ketogenic diet favorably affects serum biomarkers for cardiovascular disease in normal-weight men. J. Nutr. 132: 1879-85, 2002.
[29] Sharman M J, Gomez A L, Kraemer W J, Volek J S. Very low-carbohydrate and low-fat diets affect fasting lipids and postprandial lipemia differently in overweight men. J Nutr 2004; 134:880-5.
[30] Ruano G H T. Physiogenomics: Integrating systems engineering and nanotechnology for personalized health. In: J B, ed. The Biomedical Engineering Handbook, 2005.
[31] Huttunen J K. Physical activity and plasma lipids and lipoproteins. Ann Clin Res 1982;14 Suppl 34:124-9.
[32] Volek J S, Westman E C. Very-low-carbohydrate weight-loss diets revisited. Cleve Clin J Med 2002;69:849, 853, 856-8 passim.
[33] Westman E C, Mavropoulos J, Yancy W S, Volek J S. A review of low-carbohydrate ketogenic diets. Curr Atheroscler Rep 2003;5:476-83.
[34] Brehm B J, Seeley R J, Daniels S R, D'Alessio D A. A randomized trial comparing a very low carbohydrate diet and a calorie-restricted low fat diet on body weight and cardiovascular risk factors in healthy women. J Clin Endocrinol Metab 2003;88:1617-23.
[35] Meckling K A, Gauthier M, Grubb R, Sanford J. Effects of a hypocaloric, low-carbohydrate diet on weight loss, blood lipids, blood pressure, glucose tolerance, and body composition in free-living overweight women. Can J Physiol Pharmacol 2002;80:1095-105.
[36] Meckling K A, O'Sullivan C, Saari D. Comparison of a low-fat diet to a low-carbohydrate diet on weight loss, body composition, and risk factors for diabetes and cardiovascular disease in free-living, overweight men and women. J Clin Endocrinol Metab 2004;89:2717-23.
[37] Sondike S B, Copperman N, Jacobson M S. Effects of a low-carbohydrate diet on weight loss and cardiovascular risk factor in overweight adolescents. J Pediatr 2003;142:253-8.
[38] Yancy W S, Jr., Olsen M K, Guyton J R, Bakst R P, Westman E C. A low-carbohydrate, ketogenic diet versus a low-fat diet to treat obesity and hyperlipidemia: a randomized, controlled trial. Ann Intern Med 2004;140:769-77.
[39] Foster G D, Wyatt H R, Hill J O, et al. A randomized trial of a low-carbohydrate diet for obesity. N Engl J Med 2003;348:2082-90.
[40] Glickman S G, Marn C S, Supiano M A, Dengel D R. Validity and reliability of dual-energy X-ray absorptiometry for the assessment of abdominal adiposity. J Appl Physiol 2004;97:509-14.
[41] Hosono S, Faruqi A F, Dean F B, Du Y, Sun Z, Wu X, Du J, Kingsmore S F, Egholm M, Lasken R S (2003). Unbiased whole-genome amplification directly from clinical samples. Genome Res 13:954-64.
[42] Yan J, Feng J, Hosono S, Sommer S S (2004). Assessment of multiple displacement amplification in molecular epidemiology. Biotechniques 37:136-8, 140-3.
[43] Tang, Z. and Tracy, R. P. (2001). Candidate genes and confirmed genetic polymorphisms associated with cardiovascular diseases: a tabular assessment. J Thromb Thrombolysis 11, 49-81.
[44] OMIM (2000): Online Mendelian Inheritance in Man, OMIM (TM). McKusick-Nathans Institute for Genetic Medicine, Johns Hopkins University (Baltimore, Md.) and NCBI, NNLM (Bethesda, Md.), 2000. URL: http://www.ncbi.nlm.nih.gov/omim/
[45] Ribalta, J., Valive, J. C., Girona, J. and Masana, L. (2003). Apolipoprotein and apolipoprotein receptor genes, blood lipids and disease. Curr Opin Clin Nutr Metab Care 6, 177-187.
[46] Wong, H. and Schotz, M. C. (2002). The lipase gene family. J Lipid Res 43, 993-999.
[47] Oakes, N. D. and Furler, S. M. (2002). Evaluation of free fatty acid metabolism in vivo. Ann N Y Acad Sci 967, 158-175.
[48] Kersten, S. (2001). Mechanisms of nutritional and hormonal regulation of lipogenesis. EMBO Rep 2, 282-286.
[49] Uyeda, K., Yamashita, H. and Kawaguchi, T. (2002). Carbohydrate responsive element-binding protein (ChREBP): a key regulator of glucose metabolism and fat storage. Biochem Pharmacol 63, 2075-2080.
[50] Lee, C. H., Olson, P. and Evans, R. M. (2003). Minireview: lipid metabolism, metabolic diseases, and peroxisome proliferator-activated receptors. Endocrinology 144, 2201-2207.
[51] Nielsen, J. N. and Richter, E. A. (2003). Regulation of glycogen synthase in skeletal muscle during exercise. Acta Physiol Scand 178, 309-319.
[52] Beltowski, J. (2003). Adiponectin and resistin—new hormones of white adipose tissue. Med Sci Monit 9, RA55-61.
[53] Libby, P. (2002). Inflammation in atherosclerosis. Nature 420, 868-874.
[54] Blake, G. J. and Ridker, P. M. (2001). Novel clinical markers of vascular wall inflammation. Circ Res 89, 763-771.
[55] Rubanyi, G. M. (1991). Endothelium-derived relaxing and contracting factors. J Cell Biochem 46, 27-36.
[56] Zöllner, S. (1998): Investigations on the regulation of endothelial nitric oxide formation with emphasis on the interaction of nitric oxide and superoxide. Dissertation. Humboldt-University, Berlin: 141 pages, http://dochost.rz.hu-berlin.de/dissertationen/biologie/zoellner-stefan/PDF/Zoellner.pdf
[57] Zoellner, S., Haseloff, R. F., Kirilyuk, I. A., Blasig, I. E. and Rubanyi, G. M. (1997). Nitroxides increase the detectable amount of nitric oxide released from endothelial cells. J Biol Chem 272, 23076-2308.
[58] Hsueh, W. A. and Quinones, M. J. (2003). Role of endothelial dysfunction in insulin resistance. Am J Cardiol 92, 10J-17J.
[59] Schaffler, A.; Orso, E.; Palitzsch, K.-D.; Buchler, C.; Drobnik, W.; Furst, A.; Scholmerich, J.; Schmitz, G.: The human apM-1, an adipocyte-specific gene linked to the family of TNF's and to genes expressed in activated T cells, is mapped to chromosome 1q21.3-q23, a susceptibility locus identified for familial combined hyperlipidaemia (FCH). Biochem. Biophys. Res. Commun. 260: 416-425, 1999.
[60] Saad, M. F.; Damani, S.; Gingerich, R. L.; Riad-Gabriel, M. G.; Khan, A.; Boyadjian, R.; Jinagouda, S. D.; El-Tawil, K.; Rude, R. K.; Kamdar, V.: Sexual dimorphism in plasma leptin concentration. J. Clin. Endocr. Metab. 82: 579-584, 1997.
[61] Lowell, B. B.; S-Susulic, V.; Hamann, A.; Lawitts, J. A.; Himms-Hagen, J.; Boyer, B. B.; Kozak, L. P.; Flier, J. S.: Development of obesity in transgenic mice after genetic ablation of brown adipose tissue. Nature 366: 740-742, 1993.
[62] Sloan, J. L. & Mager, S. Cloning and functional expression of a human Na+ and Cl— dependent neutral and cationic amino acid transporter B0+. J. Biol. Chem. 274, 23740-23745 (1999).
[63] Blundell, J. E., Goodson, S. & Halford, J. C. Regulation of appetite: role of leptin in signalling systems for drive and satiety. Int. J. Obes. Relat. Metab. Disord. 25 (Suppl. 1), 29-34 (2001).
[64] Bell C G, Walley A J, Froguel P. The genetics of human obesity. Nat Rev Genet. March 2005;6(3):221-34.
[65] Reinere A, Yekutiele D, Benjamini Y: Identifying differentially expressed genes using false discovery rate controlling procedures. Bioinformatics 19:368-375 (2003).
[66] Benjamini Y, Hochberg Y: Controlling the false discovery rate: a practical and powerful approach to multiple testing. Journal of the Royal Statistical Society, Series B 57:289-300 (1995).
[67] Benjamini Y, Hochberg Y: On the adaptive control of the false discovery rate in multiple testing with independent statistics. Journal of Educational and Behavioral Statistics 25:60-83 (2000).
[68] Rosner B: Fundamentals of Biostatistics. Belmont, Calif.: Wadsworth Publishing Co. (1995).
[69] Cleveland, W S: Robust locally weighted regression and smoothing scatterplots. Journal of American Statistical Association 74, 829-836 (1979).
[70] Cleveland W S, Devlin S J: Locally Weighted Regression: An Approach to Regression Analysis by Local Fitting. Journal of the American Statistical Association Vol. 83, pp. 596-610 (1988).
[71] Hastie T, Tibshirani R. Generalized additive models. Stat. Sci. 1: 297-318 (1986).
[72] Durrleman S, Simon R. Flexible regression models with cubic splines. Statistics in Medicine 8:551-561 (1989).
[73] Steenland K, Bray I, Greenland S, Boffetta P. Empirical Bayes adjustments for multiple results in hypothesis-generating or surveillance studies. Ca Epidemiol Biomarkers Prev. 9:895-903 (2000).

1. A marker gene set comprising a plurality of single nucleotide polymorphic gene variants, wherein the presence of any one of said single nucleotide polymorphic gene variants in a human is correlated with physiological response to diet.

2. The marker gene set of claim 1, wherein said physiological response is selected from the group consisting of change in blood triglyceride level, change in blood LDL level, change in blood HDL level, and change in body mass index, and combinations thereof.

3. The marker gene set of claim 1, wherein said plurality of single nucleotide polymorphic gene variants comprise at least one single nucleotide polymorphic gene variant of a gene selected from the groups consisting of ABCB1, ACACB, ACAT1, ACHE, ADRB1, ADRB2, AKT1, AKT2, ANGPT1, APOB, APOH, APOL3, APOL4, AVEN, BDNF, CETP, CHAT, CHKB, CPT1A, CRHR2, CYP1A2, CYP2C19, CYP7A1, DBH, DRD3, DRD4, DRD5, DTNBP1, FLJ32252, FLT1, GABRA2, GAD1, GAD2, GAL, GNAO1, GYS2, HIF1A, HTR3A, ICAM1, IL10, IL1R1, INSR, IRS1, KDR, LDLR, LIPE, LIPF, LOC391530, OLR1, OXT, PIK3C2G, PIK3C3, PIK3R1, PPARG, PRKAA1, PRKAB1, RARB, RARG, RXRA, SCARB2, SELE, SSTR3, VEGF, and combinations thereof.

4. The marker gene set of claim 3, wherein said plurality of single nucleotide polymorphic gene variants comprises one or more single nucleotide polymorphic gene variants selected from the group consisting of rs10082776, rs1018381, rs1040410, rs 10422283, rs1042713, rs1042718, rs 1045642, rs10460960, rs1062688, rs1064344, rs107540, rs10841044, rs10890819, rs11212515, rs1128503, rs1150226, rs1190762, rs1290443, rs132642, rs132661, rs1478290, rs167771, rs1801278, rs1801701, rs1951795, rs2005590, rs2033447, rs2049045, rs2071710, rs2125489, rs2192752, rs2240403, rs2241220, rs2301108, rs2306179, rs2429511, rs2470890, rs2494746, rs2514869, rs2702285, rs2742115, rs2743867, rs2867383, rs3024492, rs322695, rs3750546, rs3756007, rs3757868, rs3791850, rs3791981, rs3792822, rs3808607, rs3813065, rs3853188, rs4135268, rs4244285, rs4531, rs461404, rs4802071, rs4804103, rs4986894, rs4987059, rs5030390, rs5361, rs563895, rs5880, rs5883, rs597316, rs619698, rs6265, rs694066, rs706713, rs748253, rs8110695, rs814628, rs8178847, rs8190586, rs833060, rs877172, and rs885834.

5. The marker gene set of claim 3, wherein said plurality of single nucleotide polymorphic gene variants comprises one or more single nucleotide polymorphic gene variants selected from the group consisting of rs1018381, rs10460960, rs1064344, rs1190762, rs132642, rs1951795, rs2192752, rs2306179, rs2514869, rs3024492, rs3756007, rs3757868, rs3791981, rs4244285, rs4531, rs4804103, rs4987059, rs5361, rs5883, rs8110695, rs814628, and rs877172

6. The marker gene set of claim 4, wherein said physiological response is change in blood LDL level and wherein said single nucleotide polymorphic gene variants comprises one or more single nucleotide polymorphic gene variants selected from the group consisting of rs1018381, rs4804103, or both.

7. The marker gene set of claim 4, wherein said physiological response is change in blood HDL level and wherein said single nucleotide polymorphic gene variants comprises one or more single nucleotide polymorphic gene variants selected from the group consisting of rs1064344, rs3756007, rs8110695, rs4244285, rs3024492, rs2192752, rs2514869, rs1190762, and combinations thereof.

8. The marker gene set of claim 4, wherein said physiological response is change in blood triglyceride level and wherein said single nucleotide polymorphic gene variants comprises one or more single nucleotide polymorphic gene variants selected from the group consisting of rs132642, rs3757868, rs1951795, rs3791981, rs10460960, and combinations thereof.

9. The marker gene set of claim 4, wherein said physiological response is change in body mass index and wherein said single nucleotide polymorphic gene variants comprises one or more single nucleotide polymorphic gene variants selected from the group consisting of rs814628, rs4531, rs2306179, rs4987059, rs5883, rs5361, rs877172, and combinations thereof.

10. A method for predicting an individual's physiological response to low-carbohydrate interventional dieting comprising determining if the individual has a single nucleotide polymorphic gene variant of the marker gene sets of any of claims 1-9.

11. A method for predicting an individual's physiological response to diet comprising:
A) obtaining DNA or RNA from an individual; and
B) assaying the DNA or RNA to determine the presence of one or more of the single nucleotide polymorphic gene variants of the marker gene sets of any of claims 1-9.

12. The method according to claim 11, wherein the assay performed is an array.

13. A system for predicting an individual's physiological response to diet comprising an assay for determining the presence of a single nucleotide polymorphic gene variant of the marker gene set of any of claims 1-9, said assay comprising a support material having immobilized thereon a single nucleotide polymorphic gene variant of the marker gene set of any of claims 1-9, or a subsequence thereof, and optionally comprising, nucleic acid probes, detection label, buffer, controls and instructions for use.

14. The system according to claim 13, wherein the assay is a micro- or nano-array.
